(19) United States
(12) Patent Application Publication
(10) Pub. No.: US 2012/0231527 A1
(43) Pub. Date: Sep. 13, 2012

(54) SYSTEM AND PROCESS FOR THE TREATMENT OF GAS EMISSIONS AND EFFLUENTS, AND PRODUCTION OF ALGAL BIOMASS

(75) Inventors: Nathalie Dubois-Calero, Montreal (CA); Gerard Magnin, Montreal (CA)

(73) Assignee: Alga-Labs Inc., Montreal (CA)

(21) Appl. No.: 13/395,307

(22) PCT Filed: Sep. 10, 2010
(86) PCT No.: PCT/CA10/01384

§ 371 Date: May 25, 2012

Related U.S. Application Data
(60) Provisional application No. 61/241,534, filed on Sep. 11, 2009.

(51) Int. Cl. C12S 5/00; C12M 1/00; C12N 1/12

(52) U.S. Cl. 435/257.1; 435/266; 435/289.1; 435/267

(57) ABSTRACT

The present application generally relates to a process for the reduction of gas emissions, treatment of effluents and production of algal biomass, and to a system for the reduction of gas emissions, treatment of effluents and production of algal biomass.

### CROSS-REFERENCE TO RELATED APPLICATION

This application claims the benefit of U.S. Provisional Application Ser. No. 61/241,534 filed Sep. 11, 2009, the entire contents of which are hereby incorporated by reference.

### FIELD OF THE INVENTION

The present application generally relates to process for the reduction of gas emissions, treatment of effluents and production of algal biomass, as well as to a system for the reduction of gas emissions, treatment of effluents and production of algal biomass.

### BACKGROUND ART

Plant matter has been burned for fuel since the early history of mankind. More recently, the interest for plants as a source for viable combustible materials which can be used for engine fuel has grown. On the other hand, the reduction of gas emissions from various sources and the treatment of liquid effluents are also becoming increasingly necessary and/or desirable. CO₂ bio-regeneration and treatment of liquid effluents can be advantageous by using algal biotechnology due to the production of a useful, high-value products from emitted CO₂. Production of algal biomass from reduction of emission gas is an attractive concept since algal biomass has a heating value of about 5000 kcal/kg. Algal biomass can also be turned into high quality fuel-grade oil (e.g. similar to crude oil or diesel fuel (“biodiesel”)) through biochemical conversion by known technologies. Algal biomass can also be used for gasification to produce highly flammable organic fuel gases, suitable for use in gas-burning power plants. (Reed T. B. and Gaur S. “A Survey of Biomass Gasification” NREL, 2001; hereinafter “Reed and Gaur 2001”).

### SUMMARY

In accordance with a first aspect of the invention, there is provided a process for treating effluents or gas emissions and effluents comprising the steps of:
i) providing an algae-based consortium adapted for a specific effluent to be treated, and
ii) culturing the algae-based consortium in presence of the gas emission within the specific effluent to be treated hereby producing an algal biomass and reducing the gas emission.

In accordance with another aspect of the invention, there is provided a system for treating gas emissions, effluents and produce algal biomass comprising: i) a gas emission source; ii) a cultivation pond for receiving an effluent to be treated, the cultivation pond including an inlet for receiving the specific effluent to be treated from a source outside the system and an outlet for discharging the algal biomass produced; iii) a multi-blade impeller rotatably mounted within the cultivation pond for mixing the effluent to be treated, the impeller having a vertically disposed hub, the impeller being rotatable about a longitudinal axis of the hub and including blades having a radius close to the radius of the cultivation pond; and iv) a gas sparging system supported above the multi-blade impeller within the cultivation pond, the sparging system having an inlet in fluid communication with the gas emission source for receiving the gas emission.

### BRIEF DESCRIPTION OF THE DRAWINGS

FIG. 1 is flow diagram of a system for treating gas emissions and effluents in accordance with an embodiment of the present invention;

FIG. 2 is a detailed flow diagram of ae gas cooling system of the system of FIG. 1 in accordance with an embodiment of the present invention;

FIG. 3 is perspective view of a cultivation pond of the system of FIG. 1 in accordance with an embodiment of the present invention;

FIG. 4 is a detailed flow diagram of a biomass agglomeration system of the system of FIG. 1 in accordance with an embodiment of the present invention;

FIG. 5 represents the concentration of algae (cells/ml) as a function of time (days);

FIG. 6 represents the influence of pH as a function of time (days);

FIG. 7 represents the influence of pH on the concentration of algae (cells/ml) as a function of time (days);

FIG. 8 represents the influence of wastewater on pH as a function of time (days);

FIG. 9 represents the effect of wastewater, nutrient salts or wastewater and nutrients on the concentration of algae (cells/ml) as a function of time (days);

FIGS. 10a and 10h represent the effect of wastewater, nutrient salts or wastewater and nutrients on the concentration of algae (cells/ml) as a function of time (days);

FIG. 11a represents the effect of nitrogen on the concentration of algae (cells/ml) before anaerobic digestion at pH 4 as a function of time (days);

FIG. 11b represents the effect of nitrogen on the concentration of algae (cells/ml) after anaerobic digestion at pH 7 as a function of time (days);

FIG. 11e represents the effect of nitrogen on the concentration of algae (cells/ml) before anaerobic digestion at pH 7 in diluted wastewater as a function of time (days);

FIG. 11d represents the effect of nitrogen on the concentration of algae (cells/ml) before anaerobic digestion at pH 7 as a function of time (days);

FIG. 12 represents the effect of nitrogen on the concentration of algae (cells/ml) as a function of time (days);

FIG. 13 represents the effect of nitrogen on the concentration of algae (g/L) as a function of time (days);

FIG. 14 represents the concentration of algae (g/L) as a function of time (days) with a covered pond;

FIG. 15 represents the concentration of algae (g/L) as a function of time (days) with a covered pond; and

FIG. 16 represents the concentration of algae (L) as a function of time (days) with a covered pond.

### DETAILED DESCRIPTION OF PARTICULAR EMBODIMENTS

It has been found recently that the survival resiliency of microalgae inside modern water contaminants as the result of rare spontaneous mutations (V. Lopez-Rodas et al., Resistance of microalgae to modern water contaminants as the result of rare spontaneous mutations. European Journal of Phycology (2001), 36:2:179-190) has not been exploited until now for industrial purpose. The document shows that strains of algae (or consortium) resistant to toxic wastewater may be found with an acceptable chance of success for each kind of xenobiotic agent.

The disclosure is based on the novel and unexpected observation that an algae-based consortium adapted for a specific effluent was found advantageous for treating gas emissions and effluents, and for producing algal biomass. The effluents to be treated may be used to select and purify resistant strains of algae-based consortium; the toxic properties of the effluents were found advantageous for selectively stimulate the growth of the algae strains and reduce the growth of non-photosynthetic micro-organisms.

By contrast, the known methods for the treatment of gas emissions and effluents generally use the symbiotic relationship between bacteria and algae. Bacteria are known for removing metals and toxic organic carbon. However, bacteria cannot fix CO₂ and compete with algae for the nutriments reducing the ability of algae to fix CO₂. Cultivation methods have also been limited to a few numbers of algae and thus, limited species were studied and a system for an efficient and low-energy cultivation at a relatively low cost has not been yet established. In conventional cultivation in open or semi-open ponds, contamination of culture or change in micro-organism population nature and inefficient mixing cause difficulties obtaining a stable high density algal biomass.

The present disclosure relates to the use of an algae-based consortium for the treatment of effluents and gas emissions. More specifically, the present disclosure provides an algae-based consortium adapted for a specific effluent to be treated which can live and prosper in particularly extreme conditions in terms of pH or toxic compounds levels where no contaminant element or biological predator may compete.

The present disclosure also relates to the use of a system for the treatment of gas emissions and effluents, designed to allow the production of higher quantities and quality of algal biomass and an increase of the photosynthetic efficiency by optimizing the contact of algae-based consortium with sunlight.

Photosynthesis is a process that converts carbon dioxide into organic compounds, especially sugars, using the energy from sunlight. The photosynthesis can be represented by the equation: CO₂+H₂O+light=>CH₂O+O₂ where CH₂O represents a generalized chemical formula for carbonaceous biomass.

The process and system in accordance with the present disclosure may be advantageous due to the production of a useful, high-value by-products from emitted CO₂. Production of algal biomass during combustion gas treatment for CO₂ reduction is an attractive concept since dry algae has a useful heating value of roughly around 5000 kcal/kg. Algal biomass can also be turned into high quality fuel-grade oil (e.g. similar to crude oil or diesel fuel (“biodiesel”)) through biochemical conversion by technologies known in the art. Algal biomass can also be used for gasification to produce highly flammable organic fuel gases, suitable for use in gas-burning power plants. (e.g., see Reed T. B. and Gaur S. “A Survey of Biomass Gasification” NREL, 2001; hereinafter “Reed and Gaur 2001”).

Approximately 114 kilocalories (477 kJ) of free energy are stored in algal biomass for every mole of CO₂ fixed during photosynthesis. Algae are responsible for about one-third of the net photosynthetic activity worldwide. Although photosynthesis is fundamental to the conversion of solar radiation into stored biomass, efficiencies can be limited by the reduced wavelength range of light capable of triggering photosynthesis called photosynthetic active radiations (PAR), a hand roughly 400-700 nm, which is only about half of the total solar emission in term of energy. Other factors, such as respiration requirements, efficiency of absorbing sunlight and other growth conditions can affect photosynthetic efficiencies in algal bioreactors.

For example, it is assumed that 788 microeinsteins/s/m² in average arrive at the ground level, which corresponds to 68 moles of photosynthetically active radiations (PAR) per day. In theory, 8 photons are necessary to fix one molecule of C, 68 moles/day/m2 mmoles of PAR photons can give enough chemical energy to fix 8.5 mmoles of C (102 g). Considering that half of the dry biomass is carbon, we could say that 204 g/day/m2 is the maximal productivity. There are different ways to measure the photosynthetic efficiency (PE) of a system. The number of moles of O₂ emitted can be measured with a Teflon probe and divided by the flux of light (PAR in moles or Einstein). The maximum of 12.5% and 8 to 9% for blue and red light are generally found.

PE can also be estimated by measuring calorific energy. One mole of glucose (C₆H₁₂O₆) produces 672 kcal, and CH₂O produces 672/6=112 kcal. One mole of red photons (680 nm) has an energy of 42 kcal. Height photons are needed to produce 1 molecule of CH₂O, and the maximal efficiency is 112/42×8=33% for red light. It is also said that the biomass energy is 4.25 kcal/g dry weight. For example, in Katherine, W. Australia, about 5100 Kcal/m2/day (50% PAR) is available, corresponding to 5100×0.5×0.33/4.25=198 g/m2/day (J. P. Cooper, 1970, Control of photosynthetic production, in terrestrial system, in Photosynthesis and productivity in different environment by J. P. Cooper, International biology program). The same author wrote that in the Equator, the light intensity is 382-473 cal/cm2/day, in subtropical climate, 170 kcal/cm2/year and in the north temperate region, around 478 cal/cm2/day in the mid-summer.

Carbon dioxide CO₂ is metabolized by the algae in glucose during the dark phase of photosynthesis. Glucose will be used to produce other storage compounds or as a substrate for respiration. Most algae can directly use glucose but other sources of organic carbon can be used by some micro-algae simultaneously or alternatively with CO₂, including carbohydrates, carboxylic acid, amino acids, aromatics alcohol (N. C. Tuchman et al, Differential heterotrophic utilization of organic compounds by diatoms and bacteria under light and dark conditions, Hydrobiologia, 2006, 561:167-177).

Organic carbons are extensively found in effluents from refinery, petro-chemistry, chemistry, food processing, city water treatment plants and animal husbandry farms such as pig-farms.

Some strains of Chlorophycea, mainly Chlorella, can metabolized phenols and polyphenolic aromatic compounds (PAH) (Pollio et. al., 1994. Phytochemistry, 37:1269-1272;. Pinto et. al., 2003. Biotechnol Lett., 25:1657-1659). These compounds are toxic for the majority of aquatic living beings. Laccases and phenol-oxidoreductases produced by these algae are able to catalyze the oxidation of various aromatic compounds (particularly phenols) with the concomitant reduction of oxygen to water. Those strains may be specific to the different new xenobiotic compounds.

Enzymatic reactions involved frequently need, but not always chemical energy mainly under the form of adenosine tri-phosphate (ATP) produced by photophosphorylation from adenosine di-phosphate (ADP) and light (Ogbonna J. C., Yoshizawa H. And Tanaka H Treatment of high strength organic wastewater by a mixed culture of photosynthetic microorganisms, Journal of Applied Phycology, Volume 12, Numbers 3-5, October 2000, pp. 277-284(8)).

The term “algae-based consortium” when used herein will be understood to refer to selected strains of microorganisms found in the effluents to be treated consisting of micro-organisms comprising at least one micro-alga.

The term “effluent” when used herein will be understood to refer to wastewater from industry, city, landfill, agriculture and manures from animal breeding and husbandry.

The term “algae-based consortium adapted to a specific effluent” when used herein will be understood to refer to the algae-based consortium mentioned above found in an effluent to be treated and cultured in said effluent to be treated. The term “algae-based consortium adapted to the specific effluent” also refer to an algae-based consortium selected and cultured from another site having similar effluent to be treated if no suitable algae-based consortium is found on the site containing effluent to be treated.

The term “essentially consisting of unicellular algae” when used herein will be understood to refer to an algae-based consortium composed of micro-organisms in which at least one unicellular micro-alga represents at least 60% of the algae-based consortium.

The term “algae-based consortium substantially free of bacteria” when used herein will be understood to refer to the algae-base consortium comprising less than 20% of contamining microorganisms such as bacteria.

The term “gas emission” when used herein will be understood to refer to one gas that is required or preferable to the propagation and growth of algae-based consortium. In one embodiment, the gas is carbon dioxide, and may also contain other gases that are not detrimental to the propagation, growth and survival of algae-based consortium, such as oxygen, nitrogen and other inert gases present in air.

The term “algal biomass” when used herein will be understood to refer to the amount of algae cultivated in an effluent to be treated at a given time.

The term “culture medium” when used herein will be understood to include the algae-base consortium in an effluent with nutrients. In one embodiment, the culture medium includes effluents, water, nutrients, fertilizers or hormones or combinations thereof required by the algae-based consortium for growth.

The term “light” means sunlight or artificial sources of light well known in the art of horticulture.

The term “nutrients” when used herein will be understood to include any liquid, solid or gaseous material used for the propagation and growth of algae-based consortium including organic and inorganic materials.

In accordance with an embodiment of the invention, there is provided a process for treating effluents or gas emissions and effluents comprising the steps of: providing an algae-based consortium adapted for a specific effluent to be treated, and culturing the algae-based consortium in presence of the gas emission within the specific effluent to be treated hereby producing an algal biomass and reducing the gas emission.

The first step of in the above mentioned process according to an embodiment of the disclosure is to provide an algae-based consortium. The algae-base consortium is produced by establishing first the screening parameters in function of the site characteristics where the effluent to be treated are. For example, in a plant where effluents to be treated contain amount of metals or organic carbons toxic for the majority of aquatic beings, the parameters of screenings will be the concentration of the toxic metals and organic carbons. The temperature may also be considered as a screening parameter.

Once screening parameters and values are established (for example temperature of about 35° C. to about 60° C., preferably not less than 42° C. and ten times the lethal concentration in metal depending on the metal specie), the industrial site is mapped and samplings of effluents are made at places where the ascertained values of the screening parameters are encountered. For that, 12 samples (or less in function of availability) are collected at each place.

The effluents sampled at the industrial site show before and after anaerobic digester treatment, concentrations of toxic organic carbons. The algae-base consortium found in the effluent after anaerobic digestion are identified and counted. Analysis of the content of the effluent to be treated (for example, N, P, K, Mg, Ca, Fe, Na, metals, organic compounds, etc.) is made, and from those results a culture medium, adapted to the photosynthetic algae-based consortium observed is prepared. If the identification of the algae-base consortium is not possible because of its low concentration, alteration of its shape (due to stress), a standard medium (BBM, for example) is used to favors the growth of the algae-base consortium.

To selectively increase the quantity of algae-based consortium, the algae-based consortium selected from each sample of effluent is first cultured without CO₂ to increase the pH to the higher value possible and sources of nitrogen less suitable for the growth of bacteria is added to the culture medium comprising the effluent to be treated and nutrients. Different combinations of nutrients may be tested. The algae-based consortium is selected in function of the growth rate, resistance to the toxicity of the effluent and its composition. If no suitable algae-based consortium is found in the effluent to be treated, the algae-based consortium from another industrial site with similar effluent may be used.

The algae-based consortium production for the treatment of gas emission and effluents is ready when a concentration of about 10E6 cells/mL in the algae-based consortium is obtained. The algae-base consortium is cultivated in the effluent to be treated and contacted with a gas emission source for reducing said gas emission and thereby producing algal biomass.

The gas emissions are generally vented to atmosphere after removal of suspended particulates and acids (SOx and NOx) however the temperature of these gases is generally between 100° C. and 250° C. Preferably, the gas emissions are cooled to a temperature of at least 35° C. prior to their treatment. Most preferably, the gas emissions are cooled to a temperature of about 30° C. to about 35° C. Furthermore, according to an aspect of the present disclosure the process and the system further comprise a step of cooling the gas emissions.

Preferably, the gas emissions are carbon dioxide emissions. It is contemplated that the gas emission used has a concentration of carbon dioxide of about 3% to about 15% for gas emissions produced by fossil fuel combustion such as natural gas, oil or coal, about 15% to about 30% for gas emissions produced by calcination and close to 100% for carbon dioxide separated from other gases by amine adsorption/desorption.

In one embodiment, a process in accordance with the invention is using an algae-based consortium cultured in the effluent to be treated. The process in accordance with the invention may use an algae-based consortium adapted to the specific effluent to be treated from another site if no suitable algae-based consortium is found on the site containing effluent to be treated.

In another embodiment, a system in accordance with the disclosure for improving the growth of algal biomass by increasing the contact of the consortium with light is provided.

Effluents of all types could be used as nutrient sources, thus creating a virtuous cycle in terms of environmental pollution mitigation. The effluent includes but is not limited to wastewater from animal husbandry, landfill, water treatment plants, cities, power plants, refineries, petro-chemistry plants, chemical plants, food processing and combination thereof. Preferably, the effluent is wastewater from animal husbandry or petro-chemistry plants. Most preferably, the wastewater from animal husbandry is liquid manure from pig.

The algae-based consortium is composed of micro-organisms in which at least one unicellular micro-alga represents at least 60% of the algae-based consortium. Preferably, the at least one unicellular micro-alga represents about 60% to about 95% of the algae-based consortium. Most preferably, the unicellular micro-alga represents about 95% of the algae-based consortium. The algae-based consortium is essentially comprising unicellular micro-algae.

The algae-based consortium includes but is not limited to Cyanobacteria non-environmentally problematic, non-nitrogen-fixing Cyanobacteria, Cyanobacteria found in an effluent to be treated, chlorophyta such as including euglenophyta and cryptomonades, rhodophyta, dinoflagellates, phueophyta and chrysophyta. Since the classification is changing frequently, in is contemplated that these above-mentioned terms must be understood broadly. Most preferably, the algae-based consortium is IDAC number 170709-01 filed on Jul. 17, 2009 or IDAC number 271009-01 filed on Oct. 27, 2009.

The algae-based consortium in accordance with the disclosure is cultivated in the effluent to be treated with nutrients. In a particular embodiment, nutrients include but are not limited to NaNO₃, K₂HPO₄, KH2PO₄, MgSO₄, H₂O, CaCl₂, NaCl, Fe, H₃B₃, MnCl₂, ZnSO₄, NaMoO₄, CuSO₄, Co(NO₃)₂ or mixture thereof. It is contemplated that the above-mentioned nutrients may be used in different amount and concentration without extending the scope of the disclosure.

The process and method in accordance with the disclosure are susceptible to increase productivity and reduce costs to a user. The process and system in accordance with the disclosure are susceptible to reduce the contamination of the culture medium or algae-base consortium therein from contamining microorganisms.

Until now bacteria has been generally considered more suitable to remove metals and toxic organic carbon but bacteria cannot fix CO₂ from gas emissions. The algae-based consortium has the advantage of both fixing CO₂ from gas emission and removing metals and toxic organic carbon from an effluent. Generally, bacteria compete with the alga-based consortium for the nutrients, therefore reducing the ability of the consortium to fix the CO₂. The advantage of using an algae-based consortium substantially free of bacteria is that the consortium can fix higher levels of carbon dioxide than a mixture of bacteria/algae.

Contamination may occurs in open systems and also in closed systems as well. The reasons may be that the algae-based consortium is not easily purified and/or the harsh purification conditions may not only kill the contaminants but also the algae-based consortium itself. Leakage of the system or contamination of the exhaust filters may occur, for example.

Contamination of the culture medium is not desirable nor acceptable in the claimed process and/or system and contamination by organisms other than those belonging to the initial algae-oriented consortium shall be avoided for the following reasons;
1. Algae that may cause human health or environmental damages (i.e. cyanobacteria) could grow in the culture.
2. Bacteria, or adverse algae, may compete with the selected consortium for the nutrients and reduce the capacity of the consortium to fix carbon dioxide, or may destroy the consortium itself.
3. Predators (rotifers, ciliates, daphnia, etc.) could feed on the algae and reduce the consortium production.

For example, the most extended and common industrial algal culture up today is the Spirulina culture. These types of cultures are protected from contamination by maintaining the pH around 10.

The toxic properties of the effluents to be treated are used to select and purify resistant strains of algae (or consortium). A specific culture medium is then provided to the algae (or consortium) in order to selectively stimulate the growth of the strains and decrease the non-photosynthetic micro-organisms.

It is know that high pH also inhibits bacterial growth. The increase of pH during the cultivation of the consortium results from the consortium's metabolism and may explain, without being bond to any particular theory, the decrease in bacteria population, and protection from contaminants.

The process according to an aspect of the invention may be advantageously carried out continuously and is generally carried out at a pH of at least 10 in an effluent to be treated rendering the consortium and/or the culture medium substantially free of contamining microorganisms such as bacteria. Most preferably the process is carried out at a pH of about 10 to 12.

The toxic properties of the effluents were also found advantageous for selectively stimulate the growth of the algae strains and decrease the non-photosynthetic micro-organisms.

The process conditions advantageously inhibit the growth of most bacteria and favor the growth of the algae-based consortium, allowing a higher level carbon dioxide fixation and the reduction of organic carbon in the treated effluent.

In another embodiment, a process and a method in accordance with the disclosure for water cleaning is provided. The process and the system in accordance with the disclosure is susceptible to remove metals and organic compounds with certain toxicity for the environment from the effluents to be treated. The metals that may be removed by the algae-based consortium are precious metals and a dangerous metals.

According to an embodiment, the precious metals are but not limited to gold, silver, ruthenium, rhodium, palladium, osmium, iridium or platinum.

According to an embodiment, the dangerous metals are but not limited to antimony, aluminum, arsenic, barium, cadmium, chromium, copper, iron, lead, mercury, nickel, selenium or zinc.

In another aspect of the disclosure, the process is not limited to the reduction of gas emission and the treatment of effluents. In a further embodiment, a process and a system in accordance with the disclosure for producing algal biomass is provided. It is also contemplated that the process according to the present disclosure further comprises a step of harvesting the algal biomass produced from the reduction of gas emission and treatment of the specific effluent. The consequent benefits, uses of the algal biomass harvested, include, without being limited to, applications either direct or indirect in the fields of bio-diesel production, animal feed, fertilizer, alcohol, pharmaceuticals and the like.

According to an aspect of the disclosure, the algal biomass may be harvested by flocculation. Harvesting algal biomass by flocculation method is known in the art and uses two types of flocculant agents such as chemical flocculants and natural flocculants.

According to an embodiment, the chemical flocculants are but not limited to alum, aluminium chlorohydrate, aluminium sulfate, calcium oxide, calcium hydroxide, iron(III) chloride, iron(II) sulfate, polyacrylamide, polyDADMAC, sodium aluminate or sodium silicate.

According to an embodiment, the natural flocculants are but not limited to chitosan, moringa oleifera seeds, papain, a species of Strychnos (seeds) or Isinglass.

Preferably, the flocculant is a natural flocculant. Most preferably, the natural flocculant is chitosan.

Reference will now be made to the embodiment illustrated in the drawings and described herein. It is understood that no limitation of the scope of the disclosure is thereby intended. It is further understood that the present disclosure includes any alterations and modifications to the illustrated embodiments and includes further applications of the principles of the disclosure as would normally occur to one skilled in the art to which this disclosure pertains.

According to a general aspect of the claimed invention, a system for treating gas emissions and effluents, and for producing algal biomass is described. In systems known in the art, the cultivation in open or semi-open ponds usually gives rise to contamination of culture or changes in micro-organism population nature, and inefficient mixing generally causes difficulties in obtaining a stable high density algal biomass. The system according to the general aspect of the claimed invention maximizes the light absorption of the algae cells by use of a controlled turbulent flow regime, thus enhancing the efficiency and productivity of the system with a low-energy driven agitation system.

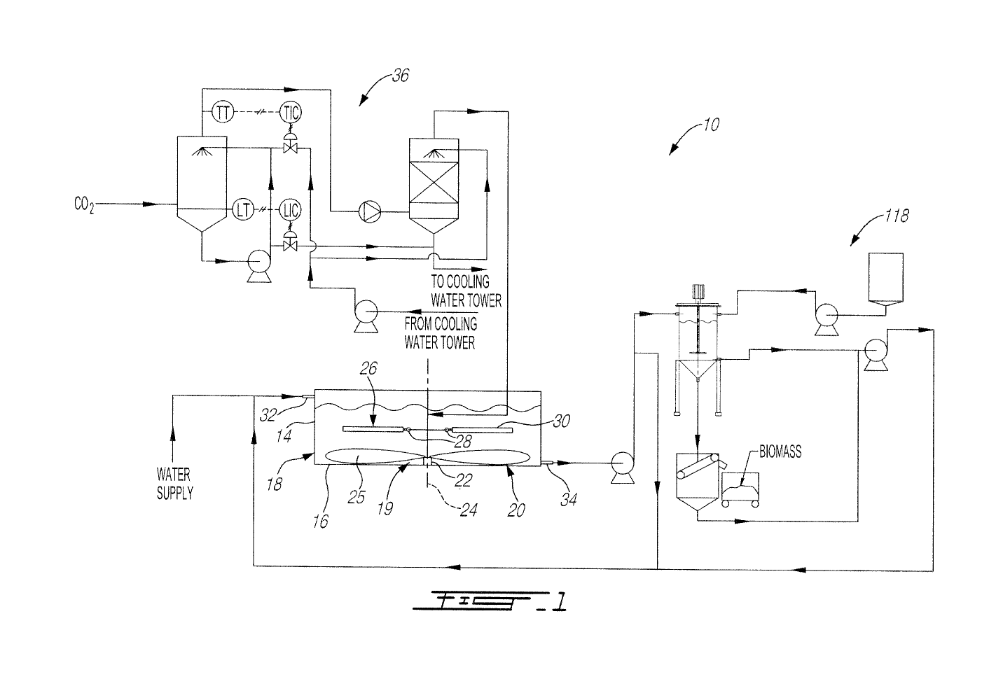

Referring to FIG. 1, a system 10 for treating gas emissions and effluents, and for producing algal biomass in accordance with a particular embodiment of the present disclosure is shown. More particularly, the system 10 comprises a gas emissions source (not shown), a gas emissions cooling system 36, a cultivation pond 18, and a biomass agglomeration system 118. The gas emissions cooling system 36 is in fluid communication with the gas emissions source for cooling the gas emissions prior to the injection of the gas emissions into the cultivation pond 18, the cultivation pound 18 is in fluid communication with the gas emission cooling system 36 for receiving the gas emissions, and the biomass agglomeration system 118 is in fluid communication with the pond 18 for harvesting the algal biomass produced therein.

The cultivation pond 18 is defined by a cylindrical sidewall 14 and a floor and receives an effluent to be treated. An agitation system 19 comprising a multi-blade impeller 20 is rotatably mounted within the cultivation pond 18 for mixing the effluent to be treated. The impeller 20 is rotatable about a longitudinal axis 24 of a vertically disposed hub 22 and blades 25 for example from three (3) to eight (8) of a radius close to the radius for example 2.5 to 25 meters of the cultivation pond 18 and rotating at low speed at a level near the floor 16. For example, the impeller is rotating at about 50 mm to about 250 mm above the floor 16 of the cultivation pond 18.

Preferably, the impeller is rotating at 100 mm above the floor 16 of the cultivation pond 18. A gas sparging system 26 is supported above the multi-blade impeller 20 within the cultivation pond 18 and has an inlet (not shown) in fluid communication with the cooling system 36 for receiving the gas emissions. For example, the gas sparging system 26 is supported at about 200 mm to about 300 mm above the multi-blade impeller 20 within the cultivation pond 18. Preferably, the gas sparging system 26 is supported at 300 mm above the multi-blade impeller 20 within the cultivation pond 18. The gas sparging system 26 comprises at least one manifold 28 and at least one diffuser 30 connected radially to the at least one manifold 28. The at least one diffuser 30 diffuses the gas emission into the specific effluent to be treated in the pond 18; at least one inlet 32 for receiving the effluent to be treated from a source outside the system and nutriments (not shown); and an outlet 34 for discharging the algal biomass produced by contacting the algae-based consortium with the gas emission and the effluent to be treated to the biomass agglomeration system 118.

As each constituent of the cultivation medium is metabolized by the algae biomass to sustain its growth, the concentration in the culture medium will decrease to a level where the specific sensor for that constituent will send a signal to the Programmable Logic Controller (PLC) indicating that addition of each constituent is required to keep its concentration in the culture media between the maximum and minimum required concentrations. A program loaded in the PLC determines the quantity of constituent solution to be added to the culture medium in the cultivation pond. The program starts the metering pump corresponding to the required constituent and keeps the metering pump running for the length of time calculated by the PLC program to add the calculated amount of constituent required. A similar liquid input system is provided for each constituent to the culture medium. In cases where two or more constituents are metabolized at the same rate, a mix solution incorporating these constituents in concentration ratios corresponding to their individual rate of metabolization by the algae biomass may be provided. This approach reduces the number of individual liquid input systems and reduces the complexity, capital cost and operating cost of the complete process. The number of liquid input systems can be as low as one and in some cases can be as much as needed. For practical reasons, the number of liquid input systems is preferably limited to six. In the case of multi cultivation pond plants, one holding tank may serve more than one pond. It can be envisaged that one single holding tank could feed each and every cultivation pond through a piping network in which the solution will be kept in movement at a velocity which will prevent settling of solids in the piping if and when suspended solids are present.

The inlet(s) 32 and outlet(s) 34 of the pond 18 include but not limited to pumps, valves and flow control systems controlled by a computer based system which adds the required quantity of effluents, chemicals, nutrients and water based on a recipe specific to the strain of algae-based consortium being cultivated in the cultivation pond 18 and from the information provided to the computer from sensors located in the pond 18 and measuring, temperature, pH, Oxidation-Reduction Potential (ORP), NO₃⁻, PO₄⁻.

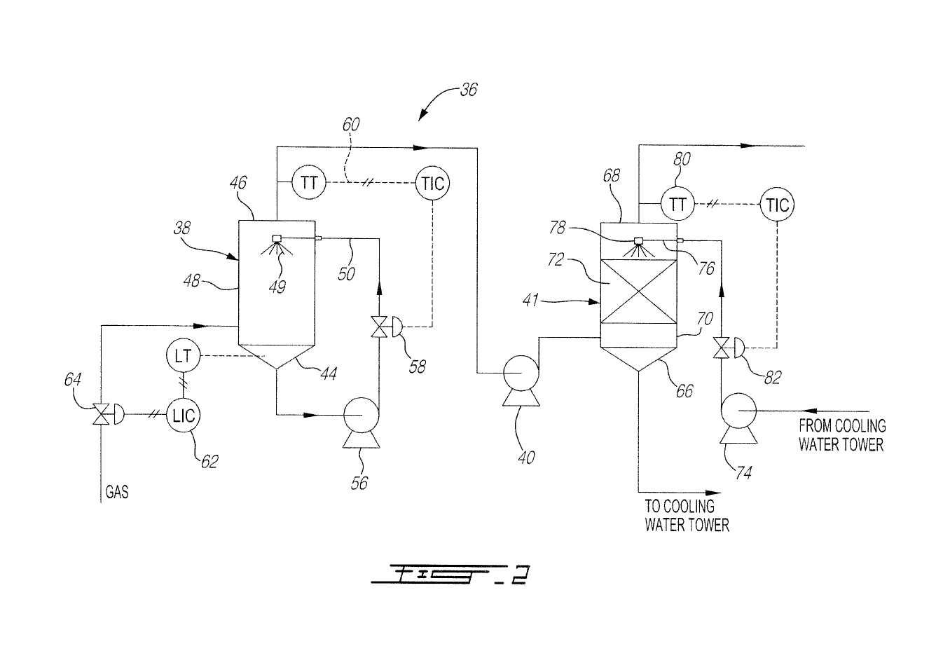

Referring to FIG. 2, the gas emission cooling system 36 according to an embodiment of the invention is shown. The cooling system 36 comprises a quench tower 38, a gas pressure blower 40, a saturation tower 41 and a sub-cooling tower (not shown).

The quench tower 38 has a base 44, a top 46 and one or more sidewall 48 extending from the base 44 to the top 46. The tower 38 includes a spray header 50 with spray nozzles 52 for spraying water located near the top 46. In an embodiment, the quench tower 38 is a cylindrical vessel having a conical base with an internal diameter calculated to have a gas velocity of between 8 and 10 feet per second. Preferably, the height of the quench tower 38 is calculated to provide a retention time of 2.5 to 3 seconds.

The water spray 49 injected in the tower 38 is produced by a water atomization system which includes a connection to the conical base 44 of the quench tower 38, a water pump 56, a flow control valve 58, a water temperature indicator/controller 60, a set of connecting piping, manual isolation valves, a spray header 50 and single phase spray nozzles 52. To compensate for the loss of water by evaporation and keep the water volume in the quench tower 38 constant a level controller 62 connected to a water flow control valve 64 adds recycled water from the agglomeration clear water circuit as water make-up.

It is contemplated that various types of spray nozzle patterns are available, can be used for the purpose of the invention and will perform similarly. The skilled person in the art would appreciate that various material of construction can be used for the construction of the quench tower without impact to the performance of the quench tower.

The gas emissions from the gas emissions source are introduced near the base 44 of the tower 38 and travel upward counter current to the water spray 49 from the spray nozzles 52. As the gas emissions travel from the base 44 of the quench tower 38 toward the top 46 and the water spray 48 falls down to the base 44 of the quench tower 38 by gravity, a thermal exchange takes place between the gas emissions and the water droplets which are heated and partially evaporated while the gas emission is cooled and saturated with humidity. The gas emissions exit the quench tower 38 at the top 46, with a relative humidity near 100%.

The injection of gas emissions into the cultivation pond requires a certain pressure to transfer the gas emissions through the gas emission cooling system 36 to the cultivation pond. Vacuum is also required to draw the gas emission from the gas emissions source through the quench tower 38.

The sum of the vacuum and pressure required to transfer the gas emissions from the source to the cultivation pond is called the total static pressure rise and is produced by a gas pressure blower 40 in fluid communication with the quench tower for transferring the gas emission to the saturation tower 41. The skilled person in the art would know that various types of gas pressure blowers exist, any type which satisfies the gas flow, total static pressure rise and humid gas handling conditions can be used without modifying the invention.

The passage of the gas emissions through the gas pressure blower 40 raises the pressure of the gas emissions but also raises the temperature of the gas emissions. In an embodiment, the pressurized gas emissions are re-saturated and cooled to the cultivation pond operation temperature which is preferably at least 35° C. and most preferably between about 30° C. and about 35° C.

As such, a saturation tower 41 is provided, having a base 66, a top 68 and a cylindrical sidewall 70 extending from the base 66 to the top 68. Preferably, the saturation tower 41 is a cylindrical vessel having an internal diameter calculated to have a gas velocity of between 8 and 10 feet per second. Preferably, he height of the saturation tower 41 is calculated to provide a retention time of 2.5 to 3 seconds.

Cooling water coming from process water cooling tower (not shown), is pumped by a pump 74 and introduced in the saturation tower 41 by a spray header 76 with spray nozzles 78 located near the top 68 of the tower 41.

The water injection system includes a connection to the cool water piping, a water pump 74, a flow control valve 82, a water temperature indicator/controller 80, a set of connecting piping, manual isolation valves, a spray header 76 and single phase spray nozzles 78.

In an embodiment, the saturation tower 41 further comprises a bed of high mass transfer structured packing 72 providing ample contact surface area to transfer heat from the gas emissions to the cooling water. The gas emission is introduced near the base 66 of the tower 41 and travel upward counter current to the cooling water percolating down through the structured packing 72.

As the gas emissions travels from the base 66 of the saturation tower 41 toward the top 68 through the water soaked structured packing 72, and the water travels down, by gravity, to the base 66 of the saturation tower 41, a thermal exchange takes place between the gas emissions and the water which is heated and partially evaporated while the gas emissions are saturated with humidity and cooled to the temperature mentioned above. The gas emissions exit at the top 68 of the saturation tower 41 with a relative humidity near 100%. A gas temperature sensor 80 measures the temperature of the saturated gas emissions and modulates the opening of the water flow control valve 82 through the computer control system to keep the gas emissions temperature within the above mentioned range. The water injected in the tower 41 comes from a process water cooling tower or any suitable cool water source.

It is contemplated that various types of spray nozzle patterns are available, can be used for the purpose of the invention and will perform similarly. It is also contemplated that various types of high mass transfer structured packing can be used without affecting the performance of the invention. The skilled person in the art would appreciate that various material of construction can be used for the construction of the saturation tower without impact to the performance of the saturation tower.

Gas emissions are generally vented to atmosphere after removal of suspended particulates and acids (SOx and NOx) however the temperature of these gases is generally between 100° C. and 250° C. Preferably, the cooling system 36 is cooling gas emissions to a temperature of at least 35° C. prior to their injection into the cultivation pond 18. Most preferably, the cooling system 36 is cooling the gas emissions to a temperature of about 30° C. to about 35° C.

It is contemplated that if there are no gas emissions to be treated on site, the same system 10 without the gas emission cooling system 36 may be used to treat effluent.

Turning to FIG. 3, the cultivation pond 18 according to a preferred embodiment of the invention is shown. The cultivation pond 18 optionally a cover system (not shown) and is designed to provide conditions for the optimum growth rate of the biomass inside the cultivation pond and the minimum usage of energy to produce that growth rate.

The multi-blade impeller 20 has four blades 25, each of a radius close for example about 50 mm to about 150 mm less to the radius of the pond and rotating at low speed at a level near the floor 16 of the cultivation pond 18. Preferably, the radius of each blades 25 is 100 mm less to the radius of the pond. The support and motion of the impeller 20 is provided from a top ledge 88 of the sidewall 14. Each impeller blade 25 has a top face 90, a bottom face (not shown) and a tip 92 suspended from a rotating ring 94 located at the top 88 of the pond 18. Each of the impeller upper blade tips 92 is connected to the rotating ring 94 via a connecting plate 98 for assisting the mixing of the culture medium. The impeller 20 is connected to a connecting hub 100 itself supported by a pivot located at the center of the pond. The rotating ring 94 of tubular cross section is supported by idle inverted cones shaped rollers 96. At least one of the roller 96 is driven by an electric motor 98 fed by a variable frequency drive controlled through the plant Programmable Logic Controller (PLC). The driven roller 96 provides the rotating motion to the rotating ring 94 which in turn provides the motion to the impeller 20 immersed in the culture medium.

It is contemplated that the idle inverted shaped rollers 96 may be made of polymer material, preferably neoprene.

In a preferred embodiment, the connecting plate 98 is rigid.

Alternatively, it is also contemplated that the support and motion of the impeller 84 may be provided from the connecting hub 100. The skilled person in the art would appreciate that various support and motion device may be provided differently for the impeller without extending the scope of the invention.

In a particular embodiment according to the invention, one or more than one cultivation pond may be used each connected in parallel to one or more gas emission cooling system.

The actual dimensions of the cultivation pond 18 will vary depending on the particular quantities of effluents to be treated. In a preferred embodiment, the cultivation pond is built in a size and shape to accommodate the process. It is further contemplated that the overall diameter of the cultivation pond 18 can be varied to accommodate the process. Preferably, the cultivation pond is circular. Preferably, the cultivation pond 18 has a diameter of at least four meters. Most preferably, the cultivation pond has a diameter of about 4 meters to about 50 meters.

It is contemplated that the cultivation pond 18 may be built from any adequate material known in the art. Those skilled in the art of treatment of toxic effluent will be able to select and built a cultivation pond having the preferred characteristics herein described.

Preferably the cultivation ponds can be built from steel, plastic panels or other suitable construction material. Most preferably, the cultivation pond is built from plastic panels.

The sidewall 14 is rigid enough to be able to support the pressure of the water contained inside the cultivation pond 18 as well as the weight of the agitation system 19, the weight of the gas sparging system 26 and optionally the forces produced by the wind blowing on a surface of a cultivating pond cover (not shown).

In a particular embodiment, the impeller blades profile has a variable pitch from the external tip of the blade to the center with the highest pitch near the center and the lowest pitch at the external tip of the blade. This variation in the pitch along the length of the blade partially compensates for the variation in linear velocity all along the length of the blade. The base rotation speed of the impeller is calculated as a function of the diameter of the cultivation pond to avoid cavitations, vibrations and other energy wastage.

For any given pond diameter the rotation speed of the impeller 84 can be varied from 0 rpm to 120% of the base rotation speed through the use of the variable frequency drive of the motor 98.

Adequate stirring of the culture medium is needed for an optimized light absorption by the algae cells and for a uniform cultivation thereof, for the following reasons: 1) a difference occurs between cultivation rates of a surface layer part and a bottom layer part of the liquid medium, 2) gases such as air and carbon dioxide must be evenly distributed in the liquid medium, 3) the light must reach all algae packets for proper maximum cultivation, 4) sedimentation of algae which would otherwise build a residual mass in the liquid bottom during cultivation must be prevented.

The cultivation of microorganisms requires the close proximity of nutrients and biomass cells. In this case close proximity is defined as approximately one tenth of the diameter of the cell. As the cell depletes the nutrients and carbon source in the close proximity of the cell, and the addition of nutrients is made at a single point of entry into a large pond, agitation is required to avoid the creation of concentration gradients near the biomass cells and non-uniform distribution of nutrients in the pond. Furthermore the photosynthesis process requires the short exposure of the biomass cells to solar radiation and periods of rest into the dark zone of the cultivation pond.

In a particular embodiment, a top layer of the culture medium having a thickness of 25 mm to 50 mm is considered the bright zone, the rest of the culture medium located below that layer is considered the dark zone. For optimum photosynthesis efficiency, each micro-organism cell must stay one unit of time in the bright zone for seven to 12 units of time in the dark zone. Most preferably, the time should be brief, in a range of about 50 to about 800 milliseconds.

The gas sparging system 26 comprises one annular manifold 28 having eight tubular membrane diffuser 30 connected radially to the manifold 28 in a plan perpendicular to the longitudinal axis of the agitator system 19; and an inlet 34 in fluid communication with the cooling system to the manifold 28 for receiving the gas emissions. The gas sparging system 26 is supported above the multi-blade impeller 20 within the cultivation pond 18 by hangers 106 fixed to the top ledge 88 of the sidewall 14. The hangers 106 comprising eight rods 108 intersecting each other at a median point 109 and mounted above the cultivation pound 18. Each rod 108 has an end portion 110 fixed to the top ledge 88 of the sidewall 14 of the pond 18 over the rotation ring 94. Each of the membrane diffusers 30 is individually connected to one of the rods with any suitable connecting means known in the art. The diffuser 30 has a plurality of pores for diffusing the gas emissions into the culture medium in the pond 18.

In an embodiment, the membrane diffusers 30 may be extending radially inwardly from the annular manifold 28, extending radially outwardly from the annular manifold 28 or a combination thereof. Preferably, the membrane diffuser 30 is extending radially outwardly from the annular manifold 28.

In a particular embodiment, the number and sizing of the membrane diffusers and the sizing of the manifold is determined by the calculation of the highest metabolization rate of carbon dioxide based on the maximum PAR at the specific site, the minimum concentration of carbon dioxide in the gas emission at the specific site and the volume of liquid in the cultivation pond.

Preferably, the membrane diffusers 30 are mounted horizontally on the manifold 28 serving as gas feeding headers, to form an horizontal gas diffusion plane from which the gas will be injected as micro-bubbles into the culture medium contained in the pond 18.

Other types of gas diffusers such as H tube spargers, disk diffusers, fritted metal spargers, have been used for the dissolution of carbon dioxide in water, however tubular membrane diffusers have demonstrated to be the most efficient and tolerant to the presence of solid particles in the gas emissions and have been selected over other methods for the purpose of this application.

In one embodiment, the gas injection system 26 is located above the agitation system 19 at a depth of 35 to 40 cm under the liquid surface to ensure optimum carbon dioxide dissolution in the culture medium and low energy consumption for the injection of the gas emissions in the culture medium.

As the gas emissions are injected intermittently as micro-bubbles through the gas injection system, a flow of gas micro bubbles will rise to the surface and entrain with them a certain quantity of liquid. This vertical movement, accompanied by eddies in the proximity of the gas/liquid raising column is compensated by a corresponding amount of liquid flow from the surface to the level of the tube diffusers. This movement of liquid and gas provides a certain level of agitation however, the liquid in the pond needs supplemental agitation to achieve the required homogeneity of mixing of gas, nutrients, biomass and culture medium, provided by the impeller.

During the photosynthesis process, dissolved carbon dioxide serves as carbon source to be metabolized by the algae-based consortium cultivated in the system and contributes to the increase of the biomass concentration.

The rate of metabolization of the dissolved carbon dioxide is related to the amount of solar energy reaching the surface of the culture medium in the cultivation pond, therefore the fixation of carbon dioxide by the algal biomass in the pond is minimal during the night, and will raise in the morning and fall again in the afternoon and evening. Excess carbon dioxide in the culture medium produces carboxylic acid that reduce the pH of the culture medium and therefore slowdown the growth rate of the algal biomass. On the other hand, a lack of carbon dioxide in the culture medium deprives the culture medium of carbon source for the photosynthesis process and ultimately also reduced the growth rate of the algal biomass. Therefore, a controlled amount of carbon dioxide dissolved in the culture medium is desirable. A carbon dioxide injection control system is contemplated to continuously measure the pH of the culture media and inject the required amount of carbon dioxide gas emissions to perform the photosynthesis part of the process.

In one embodiment, the gas emission sparging system 26 further comprises a pH sensor 114 immersed in the culture media, connected to a pH controller. The pH controller sends a signal to the PLC which integrates the data and calculates the rate of fixation of carbon dioxide in the pond. From that calculation and from the data provided by the carbon dioxide analyzer located in a gas emission duct upstream of the quench tower in the gas emission cooling system, a rate of addition of gas emissions will be derived. This information is converted into a 4-20 mA signal which is sent to a gas emission control valve 116 located on the inlet 32 feeding the cultivation pond 18 with the gas emissions. The measured gas emission flow is directed to the gas sparging system 26.

Microorganisms such as micro-algae have been cultivated for years in ponds open to atmosphere. Work done with open ponds operating with culture medium temperature in the 30° C. to 35° C. range showed that loss of water due to evaporation was high and not sustainable for industrial scale biomass production plants. Pond covers provide a barrier to the introduction of wind borne dust, debris, pollen, spores and bacteria which all have a detrimental effect on the mass cultivation of microorganisms in large ponds. Furthermore, it has been demonstrated that commercially available covers greatly reduce the incidence of contamination or even protect the culture from any contamination.

The use of transparent covers however presents some drawbacks such as a rise of the temperature under the cover, a reduction of solar energy reaching the liquid surface in the cultivation pond and an accumulation of condensation on the inner surface of the cover.

Transparent covers used in the agriculture industry, namely for the cultivation of vegetables in green-houses, have recently benefited from new developments and can now offer screens for ultra-violet radiation as well as infra-red radiation reduction. These transparent polymer sheets can block more than 90% of UV and IR and let other light frequencies through with very little reduction. Transparent covers with UV and IR barriers are also now available with condensation repellent treatment on one side.

In an optional embodiment, a pond cover system may be used. The pond cover system has a transparent polymer film with UV and IR reduction treatment as well as a condensation repellent inside the cover system. The cover is attached to the top of the sidewall of the cultivation pond using a standard commercially available heavy duty Poly Fastener system. It is also contemplated that any suitable fastener system known in the art may be used. The pressure of the gas contained in the space above the cultivation media and under the pond cover shapes the cover as a dome that provides a rigid surface to the wind and avoid flapping and premature wear of the pond cover. The dome shape also drains away the rain to the periphery of the cultivation pond.

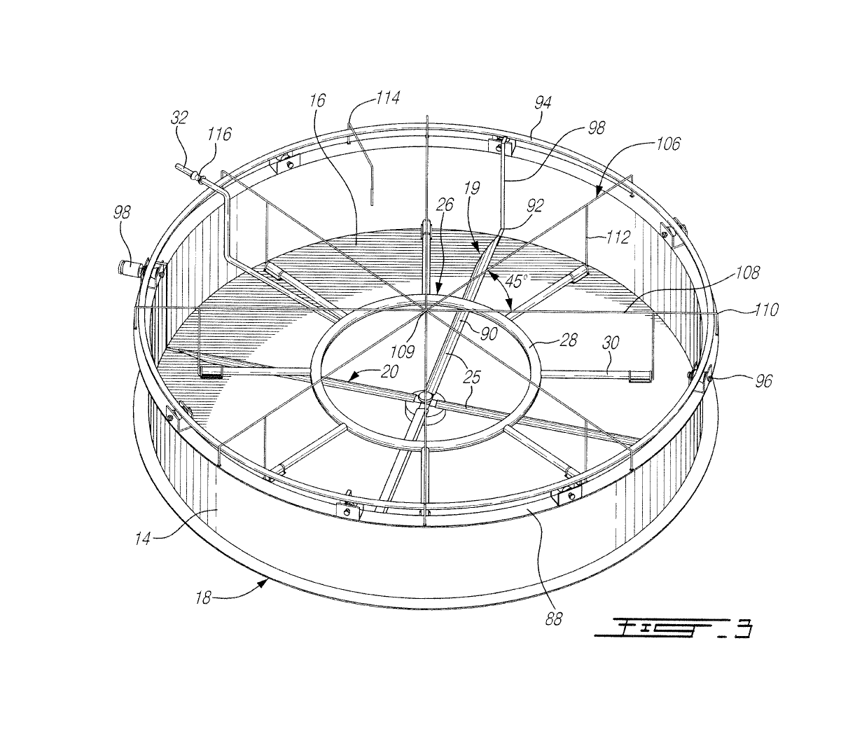

Turning to FIG. 4, the biomass agglomeration system 118 according to a preferred embodiment of the invention is shown. The biomass agglomeration system 118 comprises an agglomeration reaction tank 120, a flocculation tank 122 and a clarifier 124.

The agglomeration reaction tank 120 has a cone bottom 126, an inlet nozzle 128 with a motorized shut-off valve 130 for receiving the homogeneous suspended solution of algal biomass cultivated in the pond, an inlet 132 in fluid communication with the flocculation tank 122 for receiving a flocculent solution therein, a clear liquid drain nozzle 134 at a top 125 of the bottom cone 126 with a motorized shut-off valve 136 for draining the clear liquid, and a concentrate drain nozzle 138 located at the bottom of the cone 126 with a motorized shut-off valve 140 for draining the flocculated algal biomass (herein after the “concentrate”) to a clarifier 124.

In a preferred embodiment, the inlet nozzle 128 with a motorized shut-off valve 130 is actuated by a level control system which insures that the reaction tank does not overflow or operate at less than full volume capacity.

The concentrate is usually twenty times more concentrated than the algal biomass culture collected from the pond. For some algae strains, the concentrate can be up to 50 times more concentrated than the raw culture from the pond. These algal biomass concentrations are usually not sufficient for storage of the algal biomass prior to further treatment or commercialization and a second water removal step needs to be implemented by using centrifugation. Commercial clarifiers are used for this step as they are energy efficient as compared to other techniques known in the art such as filter presses and evaporation.

In an embodiment, a system using a plurality of culturing ponds the concentrate drained from the bottom of each of the agglomeration system reaction tanks is sent by a concentrate pump 142 to a centrally located holding tank 144 that feeds clarifiers at their design input flow capacity. The clear liquid from the clarifier is recycled to the gas conditioning system, and any excess is used as make-up water in the preparation of the nutrient solutions.

Preferably the usable volume of the tank 120 is equal to 1/168^{th} of the volume of the cultivation pond 18 and the volume of the cone 126 is equal to the volume of the settled concentrate after 20 minutes settling time. This volume is determined for each specific algae-base consortium to be cultivated in the pond.

The harvesting of the biomass from the cultivation pond is performed by draining of a fraction of the culture media from the pond and filling an agglomeration tank. The agglomeration tank operates on a one hour cycle with 24 cycles per day, seven days a week. The operation steps of the agglomeration tank include filling of the tank, addition of a chitosan flocculent, mixing of the culture with the flocculent, settling of the floc, drainage of the supernatant (clear liquid) and drainage of the floc (dark liquid). The clear liquid is recycled to the pond to keep constant the liquid level in the cultivation pond constant, the surplus clear water is used as make-up water in the gas conditioning system quench tower and as dilution water in the preparation of the nutrient solutions.

In an embodiment, the outlet 34 discharges algal biomass produced from the cultivation pond 18 on a continuous, sequential mode to the agglomeration tank.

The residence time of the algae biomass in the cultivation pond is generally three to 15 day, preferably seven day. However the skilled person in the art would understand that different residence times may be used without expending the scope of the invention.

The system and the process for treating gas emissions and effluent, and for producing algal biomass of the present application may be better understood by reference to the following examples.

### Example 1

### Treatment of Terephtalic Acid Effluents

The first step was to determine screening parameters in function of the industrial wastewater and effluents characteristics. The purified terephtalic acid (PTA) plant wastewaters before treatments by anaerobic digesters contained high amount of cobalt and manganese as well as high contents of organic carbons (terephtalic acid, benzoic acid, para-toluic acid) toxic for the majority of aquatic beings, the parameters of screenings was the concentration in the toxic metals and organic carbons.

For the screening, a concentration higher than 300 ppm of para-toluic acid was considered toxic and/or a concentration of cobalt close to 20 ppm was also considered toxic. Temperature higher than 40° C. was the last screening value.

Once screening parameters and values were determined, the site was mapped, and sampled were collected where the concentration of para-toluic acid was higher than 300 ppm and/or cobalt concentration was around 20 ppm and/or temperature was higher than 40° C. 12 samples were collected at each place.

Effluents before and after anaerobic digester treatment had toxic concentrations of para-toluic acid. Wastewaters before anaerobic digester treatment showed toxic concentrations of para-toluic acid and cobalt. Steam traps temperature was above 40° C. The micro-organisms were identified and counted. Bacteria were found in wastewaters after anaerobic digester treatments. Less than 1 cell/mL of Euglena sp. and 1 cell/mL of Chlorella-like sp were found. Their shapes were altered. No micro-organisms were found in wastewaters before anaerobic digester treatments. In steam traps, a mix of algae and bacteria was found, but the temperature was not constant. These consortiums were variable and not convenient for the process described in the disclosure.

Analysis of wastewaters (nitrite, nitrate. P, K, Mg, Ca, Fe, Na, metals, benzoic acid, terephtalic acid, para-toluic acid, etc.) were done, and from those results a culture medium adapted to the photosynthetic micro-organisms selected was prepared from a standard medium (BBM). Nitrate, phosphate, calcium and magnesium were added at different concentrations to culture medium made with 100% or 50% wastewaters treated by anaerobic digestion. The pH was higher than 7. To selectively increase the quantity of micro-algae, the first cultures were done without CO₂ at 25° C. under light.

The color of culture turned green after 7 weeks and the pH increased. The population of bacteria decreased and population of Chlorella-like algae (herein after CHX-001) increased. Few Euglena were observed. After 2 weeks, a concentration of 10E6 cells/mL was obtained in one of the treatments. The culture was stable in open flask, meaning that the consortium was not vulnerable to the contamination. The concentration in toxic elements (as previously defined) had increased in the cultures. The pH of added elements was adjusted to 8. The surviving rate was 100% after two weeks. This consortium, containing more than 95% of CHX-001, was chosen.

Cultivation of the Selected Algae-Base Consortium.

The first 50 mL of culture containing selected algae-based consortium was mixed with 200 mL of fresh culture medium. After 10 days, the 250 mL of algae-based consortium were divided in order to get 5 flasks with 50 mL of algae culture and 200 mL of fresh media.

Four treatment were done in order to determine the algae-based consortium type nutrition (autotrophy, heterotrophy or mixed type). Each treatment were replicated three times. The first treatment was made with 50% effluent after anaerobic digestier (control): 100 ml of leachate was added to BBM nutrient media in 500 ml flask, 50 ml of the algae-based consortium was also added to the flask. The flasks were shaked at 125 rpm on the shaker for 192 h, under light (90μ mol m⁻² s⁻¹).

The second treatment was made with 100% effluent after anaerobic digestier: 200 ml of leachate was added to BBM nutrient media in a flask, 50 ml of algae-based consortium was added to flask. All flasks have been shaked at 125 rpm on the shaker for 192 h, under light (90μ mol M⁻² s⁻¹).

The third treatment was made with 50% effluent+1 L CO₂/d:100 ml of leachate was added to BBM nutrient media in a 500 flask, 50 ml of algae-based consortium was also added to flask, 500 ml of CO₂/m was injected to the flask 2 times a day. All flasks have been shaked at 125 rpm on the shaker for 192 h. under light (90μ mol m⁻² s⁻¹).

The fourth treatment was made with 50% effluent+3 L CO2/d:100 ml of leachate was added to BBM nutrient media in 500 ml flask, 50 ml of algae-based consortium was added to flask, 750 ml of CO₂/30 s was injected to the flask 4 times a day. All flasks have been shaked at 125 rpm on the shaker for 192 h, under light (90μ mol m⁻² s⁻¹).

The pH for each treatment was measured daily at 16 h before adding CO₂ The pH was 7.6

The BBM nutrient media that was used is: 62.5NaNO₃, 75K₂HPO₄, 175KH₂PO₄.75MgSO₄.7H₂O, 25CaCl₂, 2H₂O, 2.5NaCl, 14Fe, 2.86H₃BO₃, 1.81MnCl2, 4H₂O, 0.222ZnSO₄.7H₂O, 0.39NaMoO₄.5H₂O, 0.0079CuSO₄.5H₂O, 0.0494 Co (NO₃)₂.6H₂O. The quantities were expressed in (mg/L).

The concentration of nitrogen (NO₃) and phosphate (PO₄) in the medium was determined on a daily basis for each treatment. The cadmium reduction method was used for nitrogen content and the orthophosphate (amino acid) method was used for the determination of the phosphate content.

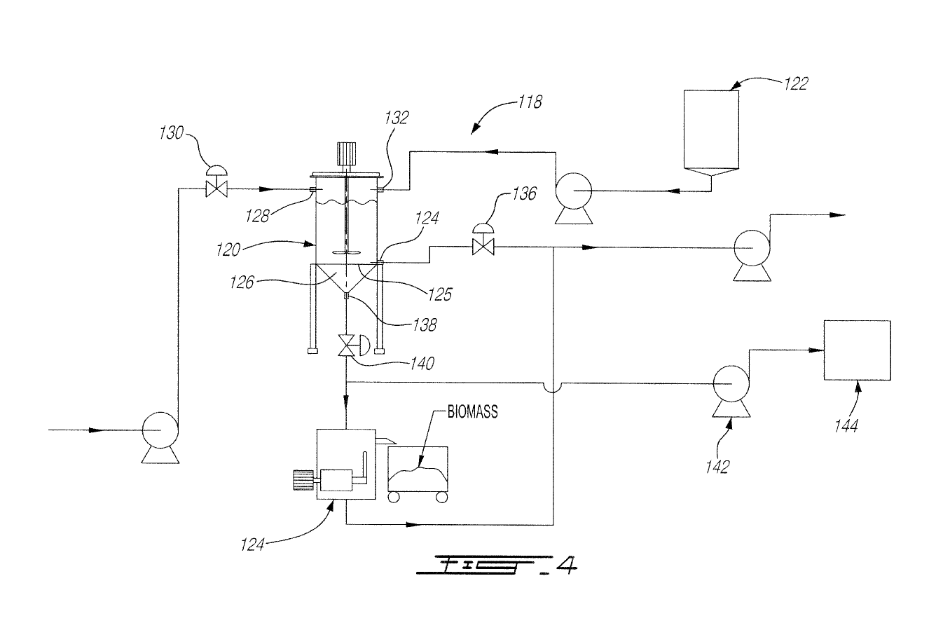

As it can be seen from FIG. 5, the algae concentration level (cells/mL/d) was higher in treatment 4 compared to the control after 7 days (2.5E+07), while CO₂ reduced the concentration level of algae after 2 days.

The dry weight (g/L) of treatment 4 was significantly higher compared to the control after 7 days of culture (0.023E), while this weight was clearly lower in both treatments 2 and 3 compared to control (Table 1).

TABLE 1
Algae Dry weight (g/L) obtained after 7
days of culture on BBM nutrient media.

| Treatments | Average | SE |
|---|---|---|
| 1—50% leachate | 0.454 | 0.062 |
| 2—100% leachate | 0.600 | 0.023 |
| 3—50% leachate + 1 L CO₂/d | 0.127 | 0.023 |
| 4—100% leachate + 3 L CO₂/d | 0.126 | 0.002 |

As it can be seen from FIG. 6, the pH was measured on the first day and decreased under 6 in both treatments with CO₂. The pH increased to about 8 on the second day until the 7^{th} day. The pH for treatments without CO₂ increased to over 11 after 7 days of assay.

The results showed that the selected consortium was in majority heterotrophy, due to the reduction of their concentration per ml when CO₂ is introduced in the culture medium. The cell number was 8 times higher for treatment of 100% effluent after digester, and 3 times higher for treatment of 50% effluent after digester. The results were confirmed with supplementary treatments conducted on two different consortium, one with 1 L CO₂/d and another without CO₂.

### Example 2

### Determination of the More Suitable Wastewater to Increase Cells Concentration

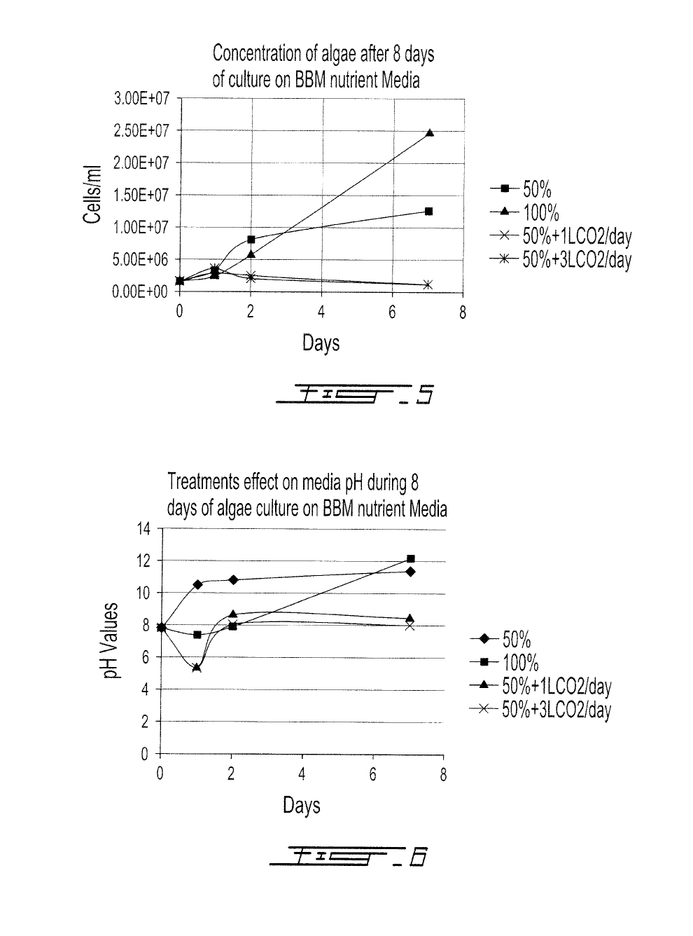

As it can be seen from FIG. 7, four different wastewaters were tested before and after anaerobic digestion. The pH of wastewater before digestion was 4, and was 7 after anaerobic digestion. Total organic carbon (TOC) in non-treated wastewater, before anaerobic digestion, was about 3000 ppm (2907-3110). TOC in treated water, after anaerobic digestion, was about 700 ppm (610-750).

The wastewaters tested were: wastewater after anaerobic digestion with addition of nutrient salts (pH 7), wastewater before anaerobic digestion with addition of nutrient salts (pH 4), wastewater before anaerobic digestion with addition of nutrient salts wherein the pH was adjusted to 7 with NaOH, and diluted wastewater (wastewater: distilled water 1:1) before anaerobic digestion with addition of nutrient salts wherein the pH has been adjusted to 7 with NaOH.

As can be seen in FIG. 7, after 8 days, cultures in effluent before digester at pH 4 showed a concentration of cells significantly lower than other treatments. When the pH was adjusted at 7, there was no difference with wastewater taken from water line after the digester.

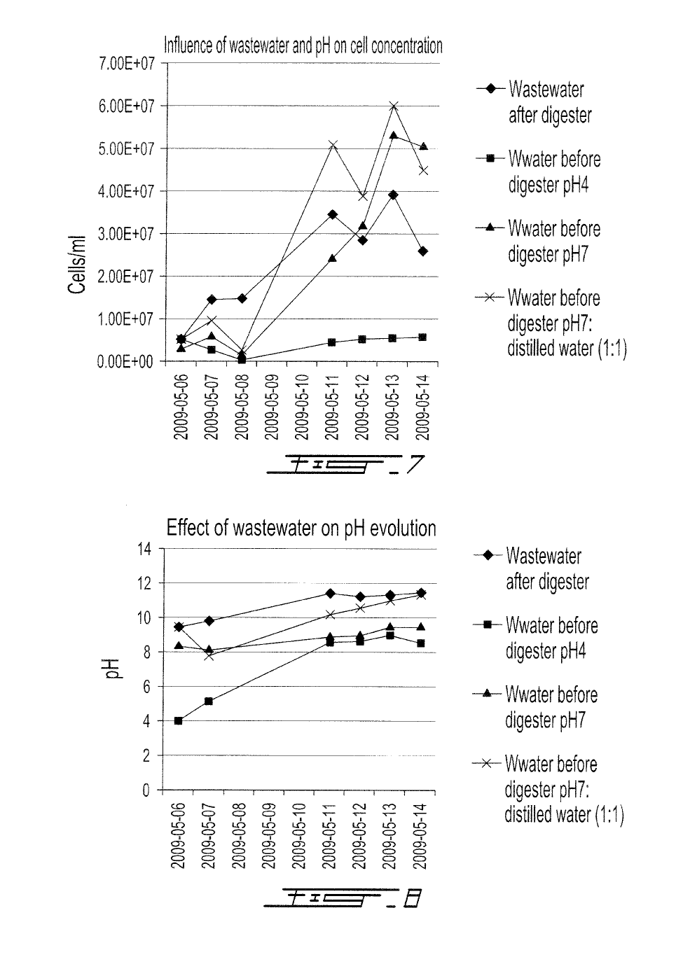

As it can be seen from FIG. 8, the pH of CHX-001 cultures raised up to 11 in wastewater after digester and diluted wastewater before digester. After 5 days, pH in the culture done in effluent at pH 4 raised up to 8.

It was concluded that both wastewater before or after digester can be used, which means that CHX-001 cultures can be used to take off the organic carbons from water and produce a valuable biomass. The high pH obtained explained why no bacteria can grow and contaminate the culture.

### Example 3

### Determination of the Time Necessary to Consume the Organic Carbon Charge of Wastewater (after Anaerobic Digestion)

Three different treatments were applied to the wastewater:

First treatment: nutrient salts were added to 200 mL of wastewater (after anaerobic digestion) and 50 mL of consortium in 500 mL flask. The flasks were shaked at 125 rpm on the shaker for 192 h, under light (90μ mol m⁻² s⁻¹).

Second Treatment: 200 ml of wastewater (after digester) and 50 ml of consortium was mixed in 500 mL-flask. The flasks were shaked at 125 rpm on the shaker for 192 h, under light (90μ mol m⁻² s⁻¹).

Third treatment: nutrient salts were added to 100 ml of consortium in 500 mL flask. The flasks were shaked at 125 rpm on the shaker for 192 μl, under light (90μ mol m⁻² s⁻¹).

Each treatment consisted of two replicates with two different consortium and the algae cells raw multiplication rate was calculated with the following equation:

(Cells concentration at day (d)−initial cells concentration)/initial cells concentration

The percentage of increase of algae dry weight (g/L) was measured during the exponential multiplication phase (day 16 to 21).

Results

Referring to FIG. 9, a 9 days latency phase was observed after adding nutrients salts and/or wastewater. After the latency phase, cell multiplication was higher in medium with low organic carbon content, where no process had been added at day 6, for both replicates. After 22 to 23 days, cells number decreased when process water was added at day 6, typical exponential curves were obtained after latency phase.

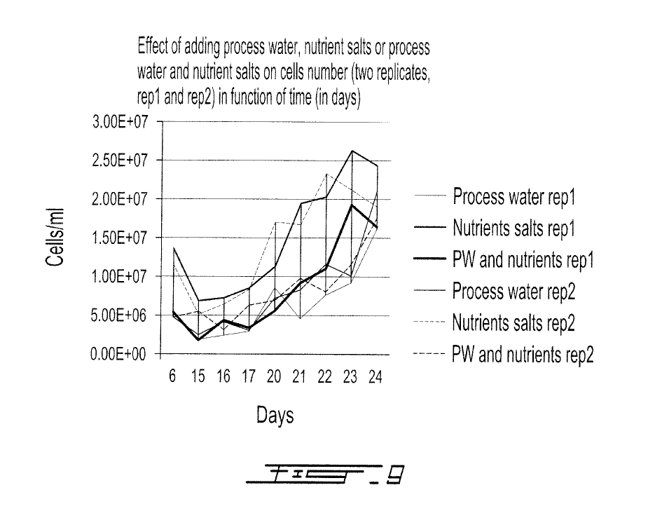

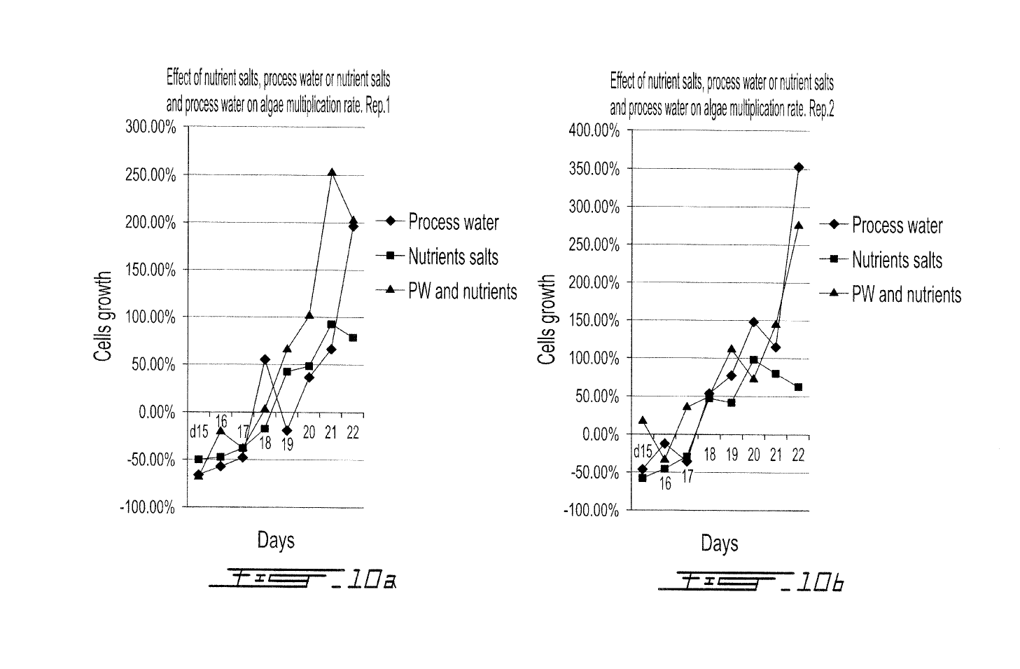

As can be seen in FIGS. 10a and 10b, cell multiplication rates of both studied algae inoculums showed that the multiplication rate of cultures in which no wastewater had been added was significantly different from the multiplication rate of cultures in which wastewater (the source of organic carbon) had been added, after day 21.

Dry weight of cultures from both consortium in which wastewater (WP) had been added (WP+nutrient salts and WP, consortium 1 and 2) were similar. Dry weight of cultures from both consortiums in which only nutrient salts had been added are higher than in other treatments. The higher dry weight obtained was 1.53 g

TABLE 2
Percentage increase of dry weight algae both algae inoculums
after being subjected to several treatments during five days.

| | | DW at day | DW at day | |
|---|---|---|---|---|
| Consortium | Treatment | 16 in g/L | 21 in g/L | Increase |
| 1 | Process water + salts | 0.29 | 0.56 | 93% |
| 1 | Process water | 0.28 | 0.44 | 58% |
| 1 | Salts | 0.42 | 1.27 | 198% |
| 2 | Process water + salts | 0.29 | 0.59 | 103% |
| 2 | Process water | 0.28 | 0.42 | 48% |
| 2 | Salts | 0.77 | 1.53 | 103 |

The content in carbon was exhausted at the 23^{rd} day. Multiplication of algae was stimulated by the wastewater addition (increase in organic carbons) and the dry weight was increased when the concentration in organic carbon was low (or when there is no fresh wastewater).

Dry weight cannot be much higher than 1.5 g/L or 750 ppm of C, because the content in organic carbon in this water process is around 1000 ppm, and some carbon is lost for respiration.

### Example 4

### Determination of the Effect of NH₄ on Algae Cultivated with Different Types of Effluents

Process waters before and after anaerobic digestion were used. The pH of wastewater before digestion is 4, and rises to 7 after anaerobic digestion.

A minimum quantity of nitrate (40 ppm of nitrogen) was put in all the 3 treatments; in treatment 1, no addition was done, in treatment 2, 40 ppm of nitrogen (nitrate) were added, in treatment 3, 40 ppm of nitrogen (NH₄) and 40 ppm of nitrogen (nitrate) were added.

Four assays were done using 1) wastewater after anaerobic digestion with nutrient salts at pH 7, 2) wastewater before anaerobic digestion with nutrient salts wherein pH was adjusted to 7 with NaOH, 3) 50% of the wastewater before anaerobic digestion and nutrient salts wherein pH was adjusted to 7 with NaOH and 4) wastewater before anaerobic with nutrient salts (pH 4). Three treatments were applied to each assay.

Treatment 1 (control): nutrient salts were dissolved in 200 mL of wastewater and 50 mL of consortium were added to the flask.

Treatment 2: Nutrient salts were dissolved in 200 mL of wastewater, 50 mL of consortium and 58.75 mg NH₄NO₃ were added to the flask.

Treatment 3: nutrient salts were dissolved in 200 mL of wastewater, 50 mL of consortium and 62.5 mg NaNO₃ were added to the flask.

All treatments flasks were shaked at 125 rpm on the shaker for 14 days, under light (90μ mol m⁻² s⁻¹). Each treatment consisted of 3 replicates.

TABLE 3
Dry weight

| | | | Days of culture | | | |
|---|---|---|---|---|---|---|
| | | | 0 | 6 | 8 | 14 |
| Source of | | Initial | DW | DW | DW | DW |
| nitrogen | wastewater treatment | pH | g/L | g/L | g/L | g/L |
| NO₃ | After digester | 7 | 0.17 | 0.8 | 0.87 | 1.57 |
| NH₄ + NO₃ | After digester | 7 | 0.17 | 0.87 | 0.81 | 1.59 |
| control | After digester | 7 | 0.17 | 0.825 | 0.76 | 1.435 |
| NO₃ | Before digester | 4 | 0.17 | 0.745 | 0.445 | 1.265 |
| NH₄ + NO₃ | Before digester | 4 | 0.17 | 0.545 | 0.52 | 1.51 |
| control | Before digester | 4 | 0.17 | 0.51 | 0.28 | 1.075 |
| NO₃ | Before digester | 7 | 0.17 | 1.655 | 1.52 | 3.142 |
| NH₄ + NO₃ | Before digester | 7 | 0.17 | 1.595 | 1.525 | 1.82 |
| control | Before digester | 7 | 0.17 | 1.27 | 1.295 | 1.595 |
| NO₃ | Before digester | 7 | 0.17 | 1.325 | 1.485 | 1.75 |
| | diluted | | | | | |
| NH₄ + NO₃ | Before digester | 7 | 0.17 | 1.425 | 1.43 | 1.55 |
| | diluted | | | | | |
| control | Before digester | 7 | 0.17 | 1.52 | 1.66 | 1.725 |
| | diluted | | | | | |

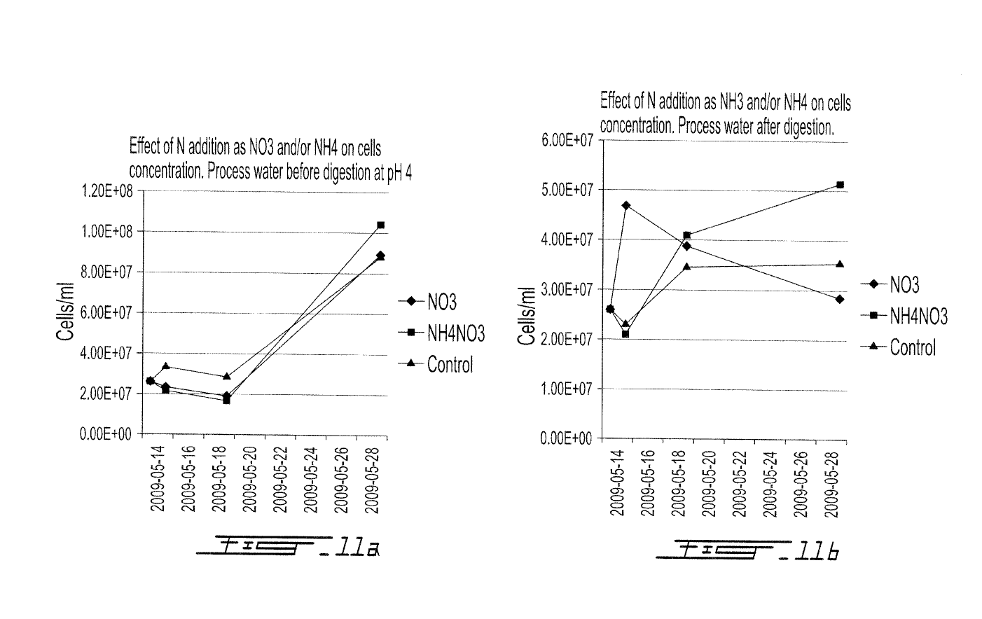

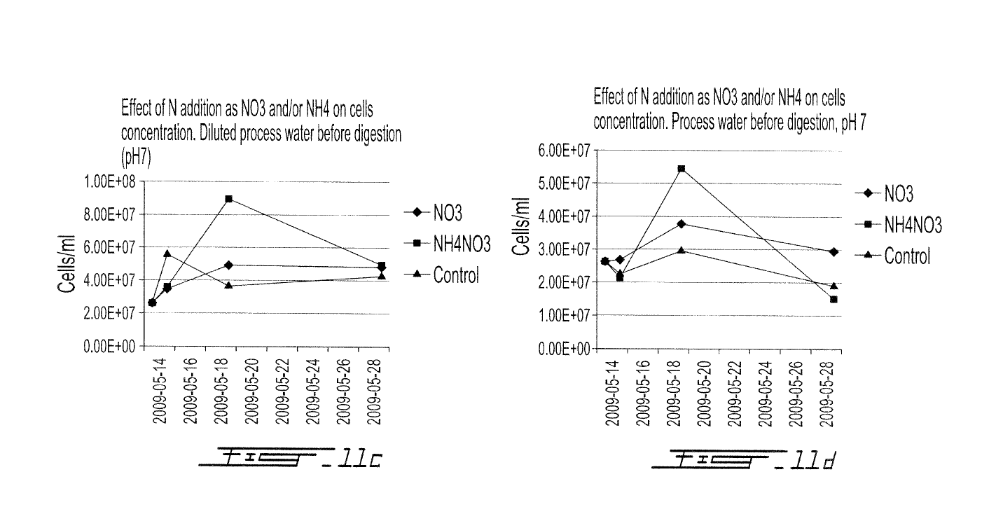

As it can be seen in FIG. 11 a to d, the dry weight after 14 days was higher when cultures were grown on process waters taken before anaerobic digestion with pH being adjusted to 7, and only nitrate being added. After 6 days, cells concentration was higher in cultures grown in wastewaters before anaerobic digestion, diluted or not with pH being adjusted to 7, and with NH₄NO₃ being added. As it can be seen in FIG. 11 c and d, cell concentration decreased after 3 days. As it can be seen in FIG. 11 a and b, the highest cell concentration was obtained after 14 days in cultures grown in wastewaters before anaerobic digestion (pH 4), and with NH₄NO₃.

Total organic carbon were reduced in non-treated wastewater (before anaerobic digestion) with pH 7 and with NO₃, from 3110 ppm to 363 ppm after 14 days. TOC were reduced from 750 ppm to 100 ppm in treated wastewater, with no addition (control)

It was concluded that the consortium CHX-001 may use nitrogen source such as nitrate or ammonium forms.

It was observed that NO₃ and neutral initial pH promoted increase in dry weight and NO₃ and NH₄ associated with low initial pH, increase the cells concentrations.

The best growth is calculated using the dry weight or the cell concentrations found in cultures grown in wastewater taken before digester, in which the organic carbon content is higher. TOC were reduced by 90% in 14 days or less.

### Example 5

### Determination of the NO₃ Concentration Inducing High Growth of CHX-001

40 ppm, 120 ppm, 240 ppm of N—NO₃ or 60 ppm N—NO₃ with 60 ppm N—NH₄ were added to non-treated wastewater with nutrient salts, in triplicates.

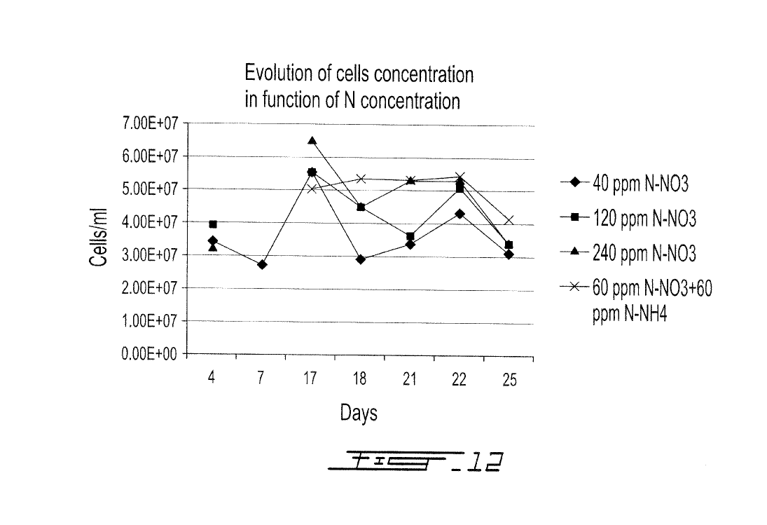

As it can be seen in FIG. 12, higher cells concentration were obtained after 17 days of culture. Afterward, cell concentration decreased for treatments with 40 ppm and 120 ppm N—NO3 and 40 ppm N—NO3. Cultures with nitrogen such as NO₃ and NH₄ gave high and stable concentration in cells.

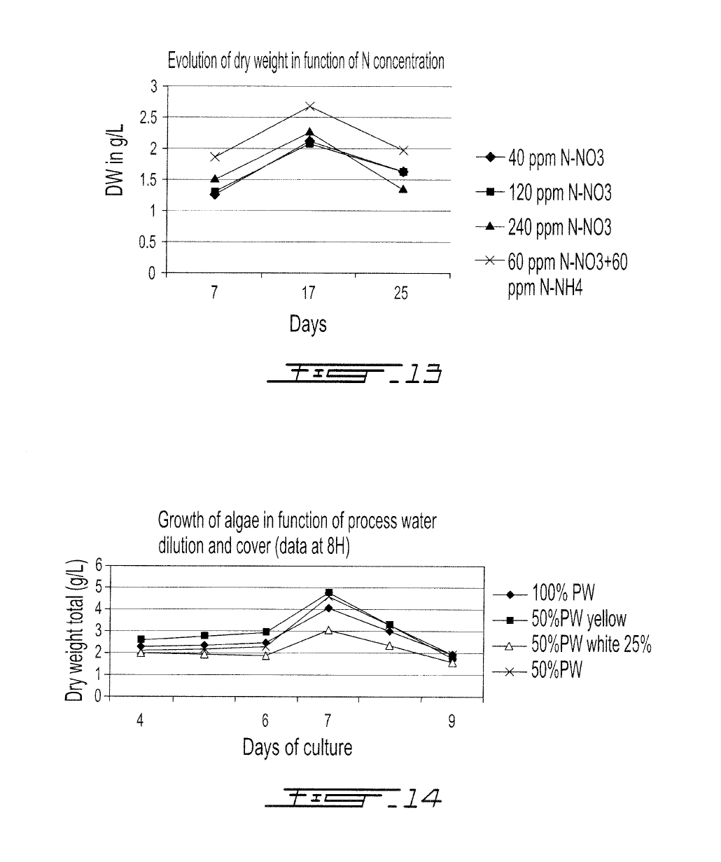

As it can be seen in FIG. 13, dry weight gave similar results: nitrogen such as nitrate and ammonium source induced higher dry weight.

It was concluded that a mixture of nitrogen sources such as NO₃ and NH₄ induced higher dry weight and cell concentration in non-treated wastewater.

### Example 6

### Use of Covers

Ponds may be covered to avoid water evaporation, algal propagules dispersion and, CO₂ dispersion if used. In order to determine the type of plastic that allowed the maximal growth of algae in tropical climate, algae were grown in 350 L tanks under natural light.

Tank 1 was filled up with 305 L process water (after anaerobic digestion) and 35 L inoculums, without cover; tanks 2, 3 and 4 were filled up with 152.5 L process water (after anaerobic digestion), 152.5 L of distilled water and 35 L inoculums. The same quantity of nutrients were added in each tank.

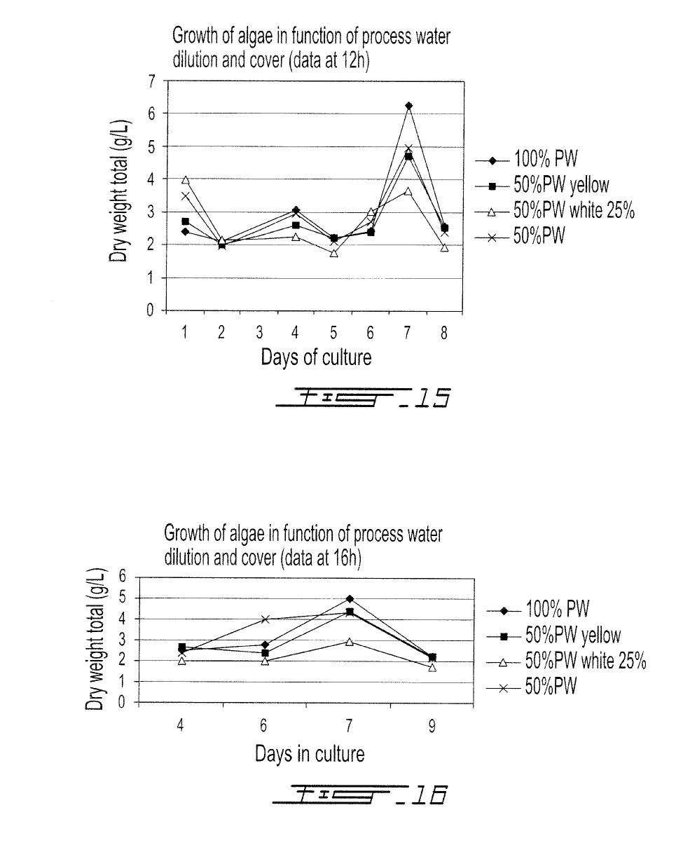

Tank 2 was covered with a yellowish plastic that stops UV radiations and a part of violet radiations; tank 3 was covered with a whitish plastic that stops 25% of light and tank 4 was not covered (FIGS. 14 to 16).

Data on dry weight were taken at 8.00 AM, 12.00 AM and 4.00 PM every day during 9 days.

Concentration of algae (or consortium) is generally higher at midday, when the light intensity is higher. The nutrients and/or organic carbon from wastewater were exhausted after 7 days. The results are illustrated in FIGS. 14 to 16.

### Example 7

### Agglomeration with Chitosan

Different concentration of chitosan solution were used to flocculate culture medium having the same concentration

Experiments:

Test 1:

Date: 27 Mar. 2008

Culture: C4

Dry Weight: 1.94 g/l

Cell Concentration: 1.70E+08

PH: 6.80

| ITEM | C4 | CHITOSAN | RESULTS |
|---|---|---|---|
| 1 | 200 ml | 3 ml (50%) | 10 minutes after mixing, floccules |
| | | | were found, liquid became clear |
| 2 | 200 ml | 1 ml (50%) | 10 minutes after mixing, floccules |
| | | | were found, liquid became very clear |
| 3 | 200 ml | 3 ml (100%) | 10 minutes after mixing, a few floccules |
| | | | were found, liquid was not clear |
| 4 | 200 ml | 1 ml (100%) | 10 minutes after mixing, floccules |
| | | | were found, liquid became clear |
| Control | 200 ml | no | After 10 minutes, no floccules found. |

Item 2 with the culture medium having a concentration of 1.94 g/l flocculated with 1 ml 50% chitosan solution gave the most satisfactory result.

Test 2:

Date: 31 Mar. 2008

Culture: C4

Dry Weight: 1.24 g/l

Cell Concentration: 7.68E+07

PH: 6.30

| ITEM | C4(ml) | CHITOSAN | RESULTS |
|---|---|---|---|
| 1 | 200 | 1 ml (60%) | 10 minutes after mixing, some floccules |
| | | | were found, liquid became clear |
| 2 | 200 | 1 ml (50%) | 10 minutes after mixing, some floccules |
| | | | were found, liquid became clear |
| 3 | 200 | 1 ml (40%) | 10 minutes after mixing, some floccules |
| | | | were found, liquid became clear |
| 4 | 200 | 1 ml (30%) | 10 minutes after mixing, more floccules |
| | | | were found, liquid became very clear |
| 5 | 200 | 1 ml (20%) | 10 minutes after mixing, more floccules |
| | | | were found, liquid became clear |
| 6 | 200 | 1 ml (10%) | 10 minutes after mixing, few floccules |
| | | | were found, liquid became a little clear |
| 7 | 200 | 1 ml (5%) | 10 minutes after mixing, few floccules |
| | | | were found, liquid became a little clear |
| Control | 200 | 0 | After 10 minutes, no floccules found. |

Item 4 with the culture medium having a concentration of 1.24 g/l flocculated with 1 ml 30% chitosan solution gave the most satisfactory result.

Test 3:

Date: 7 Apr. 2008

Culture: C4

Dry Weight: 0.86 g/l

Cell Concentration: 6.90E+07

PH: 7.11

| ITEM | C4(ml) | CHITOSAN | RESULTS |
|---|---|---|---|
| 1 | 200 | 1 ml 50% | 10 minutes after mixing, some floccules |
| | | | were found, liquid was not very clear |
| 2 | 200 | 1 ml 40% | 10 minutes after mixing, some floccules |
| | | | were found, liquid was not very clear |
| 3 | 200 | 1 ml 30% | 10 minutes after mixing, many floccules |
| | | | were found, liquid was clear |
| 4 | 200 | 1 ml 20% | 10 minutes after mixing, many floccules |
| | | | were found, liquid was more |
| | | | clear than the others |
| 5 | 200 | 1 ml 10% | 10 minutes after mixing, some floccules |
| | | | were found, liquid was not very clear |
| 6 | 200 | 1 ml 5% | 10 minutes after mixing, some floccules |
| | | | were found, liquid was not very clear |
| Control | 200 | 0 | After 10 minutes, no floccules found. |

Item 4 with the culture medium having a concentration of 0.86 g/l flocculated with 1 ml 20% chitosan solution gave the most satisfactory result.

Test 4:

Date: 24 Apr. 2008

Culture: C4

Dry Weight: 0.50 g/l

Cell Concentration: 4.60E+07

PH: 6.62

| ITEM | C4(ml) | CHITOSAN | RESULTS |
|---|---|---|---|
| 1 | 200 | 1 ml 30% | 10 minutes after mixing, some floccules |
| | | | were found, liquid was clear |
| 2 | 200 | 1 ml 20% | 10 minutes after mixing, some floccules |
| | | | were found, liquid was clear |
| 3 | 200 | 1 ml 10% | 10 minutes after mixing, many floccules |
| | | | were found, liquid was clear |
| 4 | 200 | 1 ml 5% | 10 minutes after mixing, many floccules |
| | | | were found, liquid was more |
| | | | clear than the others |
| 5 | 200 | 1 ml 4% | 10 minutes after mixing, some floccules |
| | | | were found, liquid was clear |
| 6 | 200 | 1 ml 2% | 10 minutes after mixing, some flocculcs |
| | | | were found, liquid was not clear |
| Control | 200 | 0 | After 10 minutes, no floccules found. |

Item 4 with the culture medium having a concentration of 0.50 g/l flocculated with 1 ml 5% chitosan solution gave the most satisfactory result.

Test 5:

Date: 13 May 2008

Culture: Scenedesmus from tank 2

Dry Weight: 0.48 g/l

Cell Concentration: 5.05E+06

PH: 5.31

| ITEM | S1 | CHITOSAN | RESULTS |
|---|---|---|---|
| 1 | 200 ml | 1 ml (60%) | 10 minutes after mixing, a few floccules |
| | | | were found, liquid was not clear |
| 2 | 200 ml | 1 ml (50%) | 10 minutes after mixing, a few floccules |
| | | | were found, liquid was not clear |
| 3 | 200 ml | 1 ml (40%) | 10 minutes after mixing, a few floccules |
| | | | were found, liquid was not clear |
| 4 | 200 ml | 1 ml (30%) | 10 minutes after mixing, a few floccules |
| | | | were found, liquid was not clear |
| 5 | 200 ml | 1 ml (20%) | 10 minutes after mixing, a few floccules |
| | | | were found, liquid was not very clear |
| 6 | 200 ml | 1 ml (10%) | 10 minutes after mixing, many floccules |
| | | | were found, liquid became clearer |
| 7 | 200 ml | 1 ml (5%) | 10 minutes after mixing, many floccules |
| | | | were found, liquid became more clear |
| 8 | 100 ml | 0.5 ml (5%) | 10 minutes after mixing, many floccules |
| | | | were found, liquid became more clear |
| Control | 200 ml | no | After 10 minutes, no floccules found. |

Item 7 and 8 with the culture medium having a concentration of 0.48 g/l flocculated with 5% chitosan solution gave the most satisfactory result.

Test 6:

Date: 2 Jun. 2008

Culture: Scenedesmus

Dry Weight: 2.58 g/l

Cell Concentration: 1.95E+07

PH: 6.80

| ITEM | S1 | CHITOSAN | RESULTS |
|---|---|---|---|
| 1 | 200 ml | 1 ml (60%) | 10 minutes after mixing, some floccules |
| | | | were found, liquid was more clear |
| 2 | 200 ml | 1 ml (50%) | 10 minutes after mixing, some floccules |
| | | | were found, liquid was clear |
| 3 | 200 ml | 1 ml (40%) | 10 minutes after mixing, some floccules |
| | | | were found, liquid was clear |
| 4 | 200 ml | 1 ml (30%) | 10 minutes after mixing, some floccules |
| | | | were found, liquid was clear |
| 5 | 200 ml | 1 ml (20%) | 10 minutes after mixing, some floccules |
| | | | were found, liquid was clear |
| 6 | 200 ml | 1 ml (10%) | 10 minutes after mixing, a few floccules |
| | | | were found, liquid was not clear |
| Control | 200 ml | no | After 10 minutes, no floccules found. |

Item 1 with the Scenedesmus culture medium having a concentration of 2.58 g/l flocculated with lint 50% chitosan solution gave the most satisfactory result.

Test 7:

Date: 11 Jun. 2008

Culture: C4

Dry Weight: 1.48 g/l

Cell Concentration: 1.38E+07

PH: 8.16

| ITEM | S1 | CHITOSAN | RESULTS |
|---|---|---|---|
| 1 | 200 ml | 1 ml (60%) | After 10 minutes, no floccules found. |
| | | | Same as control. |
| 2 | 200 ml | 1 ml (50%) | After 10 minutes, no floccules found. |
| | | | Same as control. |
| 3 | 200 ml | 1 ml (40%) | After 10 minutes, no floccules found. |
| | | | Same as control. |
| 4 | 200 ml | 1 ml (30%) | After 10 minutes, no floccules found. |
| | | | Same as control. |
| 5 | 200 ml | 1 ml (20%) | After 10 minutes, no floccules found. |
| | | | Same as control. |
| 6 | 200 ml | 1 ml (10%) | After 10 minutes, no floccules found. |
| | | | Same as control. |
| Control | 200 ml | no | After 10 minutes, no floccules found. |

At pH over 8, the chitosan solution cannot flocculate algae.

It was concluded that the chitosan can flocculate both C4 and Scenedesmus, the high concentration cultures need high concentration chitosan solution and the pH is an important factor of flocculation.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and as may be applied to the essential features hereinbefore set forth, and as follows in the scope of the appended claims.

1. A process for treating effluents or gas emissions and effluents comprising the steps of:
i) providing an algae-based consortium adapted for a specific effluent to be treated, and
ii) culturing the algae-based consortium in presence of the gas emission within the specific effluent to be treated thereby producing an algal biomass and reducing the gas emission.

2. The process according to claim 1, further comprising a step of harvesting the algal biomass produced from the reduction of gas emission and treatment of the specific effluent.

3. The process according to claim 1, wherein the algae-based consortium is essentially consisting of unicellular algae.

4. The process according to claim 1, wherein the algae-based consortium is substantially free of bacteria.

5. The process according to claim 1, wherein the algae-based consortium comprises the biological deposit IDAC number 170709-01 deposited on Jul. 17, 2009.

6. The process according to claim 1, wherein the algae-based consortium comprises the biological deposit IDAC number 271009-01 deposited on Oct. 27, 2009.

7.-14. (canceled)

15. The process according to claim 1, further comprising removing metals.

16.-18. (canceled)

19. The process according to claim 1, wherein the process is carried out at a pH of at least 10 in an effluent to be treated.

20.-24. (canceled)

25. An algae-based consortium comprising the biological deposit IDAC number 170709-01 filed on Jul. 17, 2009.

26. An algae-based consortium comprising the biological deposit IDAC number 271009-01 filed on Oct. 27, 2009.

27. A system for treating gas emissions, effluents and produce algal biomass comprising:
a gas emission source;
a cultivation pond for receiving an effluent to be treated, the cultivation pond including an inlet for receiving the specific effluent to be treated from a source outside the system and an outlet for discharging the algal biomass produced; a multi-blade impeller rotatably mounted within the cultivation pond for mixing the effluent to be treated, the impeller having a vertically disposed hub, the impeller being rotatable about a longitudinal axis of the hub and including blades having a radius close to the radius of the cultivation pond;
a gas sparging system supported above the multi-blade impeller within the cultivation pond, the sparging system having an inlet in fluid communication with the gas emission source for receiving the gas emission.

28.-31. (canceled)

32. The system according to claim 27, wherein the gas sparging system comprises at least one manifold and at least one diffuser connected radially to the manifold, the diffuser having a plurality of pores for diffusing the gas emission into the effluent to be treated.

33. The system according to claim 27, wherein each blade has a tip connected to a rotating ring located at a top of the sidewall of the pond and driving a rotation of the impeller.

34. The system according to claim 32, wherein the rotating ring being supported by rollers with at least one of the rollers being driven by a motor.

35. The system according to claim 27, wherein the gas sparging system is supported above the multi-blade impeller within the cultivation pond by hangers fixed on the side wall.

36. (canceled)

37. (canceled)
